SEQUENCE LISTING (8 sequences)

SEQ ID NO: 1 - DNA, 20 bp, Artificial Sequence
  Primer
cccttgaaaagcccaaacac

SEQ ID NO: 2 - DNA, 19 bp, Artificial Sequence
  Primer
ccgggcctgattgtacttc

SEQ ID NO: 3 - DNA, 22 bp, Artificial Sequence
  Primer
aggaagcttccaacgctggagt

SEQ ID NO: 4 - DNA, 20 bp, Artificial Sequence
  Primer
caagaggaatttgccgacgg

SEQ ID NO: 5 - DNA, 22 bp, Artificial Sequence
  Primer
tgttcatctctggctgtcatcg

SEQ ID NO: 6 - DNA, 21 bp, Artificial Sequence
  Primer
cctgacgtgatcctttacggt

SEQ ID NO: 7 - DNA, 20 bp, Artificial Sequence
  Primer
gagcgcggttacagcttcac

SEQ ID NO: 8 - DNA, 21 bp, Artificial Sequence
  Primer
actcttgcttcgagatccaca

(19) United States
(12) Patent Application Publication
(10) Pub. No.: US 2011/0214191 A1
(43) Pub. Date: Sep. 1, 2011

(54) METHOD FOR THE IDENTIFICATION OF GENES INVOLVED IN NEURODEGENERATIVE PROCESSES

(76) Inventors: Maria Fernanda Ceriani, Ciudad Autonoma de Buenos Aires (AR); Carolina Rezaval, Ciudad Autonoma de Buenos Aires (AR); Jimena Berni, Ciudad Autonoma de Bueno Aires (AR)

(21) Appl. No.: 13/060,211

(22) PCT Filed: Aug. 20, 2009
(86) PCT No.: PCT/IB2009/053681

§ 371 Date: Mar. 28, 2011

(30) Foreign Application Priority Data
Aug. 27, 2008 (AR) P080103729

(51) Int. Cl. A61K 49/00; C12Q 1/68; A01K 67/033

(52) U.S. Cl. 800/3; 435/6.11; 800/9

(57) ABSTRACT

A method for the identification of genes involved in neurodegenerative processes, detectable by the late onset of a phenotype associated with neurodegeneration, by means of a genetic screen of deregulated genes, which comprises the measurement of sleep-wake cycle activity schemes in different stages of life, young and adult, of individuals of an animal model, such as Drosophila. A mutant fly whose genome comprises a disruption in its enabled gene, with decrease of the enabled gene expression, and exhibiting a late onset neurodegenerative phenotype in adulthood.

### FIELD OF THE INVENTION

The present invention refers to a method for the identification of genes involved in neurodegenerative processes, particularly those related with human neurodegenerative diseases characterized by a late onset and progressive degeneration, such as Alzheimer's disease, Parkinson's disease and Huntington's disease.

### BACKGROUND OF THE INVENTION

Age is a major risk factor for neurodegenerative diseases such as Alzheimer's disease (AD), Parkinson's disease (PD) and Huntington's disease (HD), all of them representing a terrible human toll. Recent estimates claim that about 25 million people worldwide suffer from these devastating diseases, and these figures will double every 20 years to reach 81 millions by 2040 [Ferri, C. P., et.al. (2005) Lancet 366, 2112: 2117.]. In the United States alone, there are more than 5 million people affected with AD, and it is expected that this number will increase to 16 million by 2050, while there are at present more than 1 million suffering from PD.

Neurodegenerative diseases require intense and prolonged care of those affected, thereby posing a heavy burden on the population as well as social security systems.

As life expectation is extended and society ages, this type of devastating diseases will become increasingly frequent. In fact, it is expected that the proportion of individuals older than 60 years of age will double in the next 50 years. These startling statistics clearly highlight the need for thoroughly understanding the basic cellular and molecular processes underlying these disabling disorders.

Many neurodegenerative diseases share a number of characteristics such as relentless progression, late onset, association with deposits of misfolded proteins in the form of inclusion bodies, amyloid plaques or neurofibrilar tangles, which may reside in the nucleus (HD), the cytoplasm (PD), or the extracellular matrix (AD) [Ross C A et.al., (2004) Protein aggregation and neurodegenerative disease. Nat Med 10 Suppl: S10-S17]. Albeit the type of protein involved in each disease varies, the molecular and cellular mechanisms, like the formation and accumulation of cellular deposits, could hold the key to unlocking the cause of many such ailments.

The non-human animal model of Drosophila has been a highly used organism for the study of a variety of human disorders. Fortini et.al. (2000) performed an in silico search for identifying Drosophila homologous genes to those which cause diseases in humans [J.Cell Biol. 50 (2): F23. 2000]. Out of 287 human genes known to be mutated, altered, amplified or deleted in subjects with a disease, they identified 178 (amounting to 62%) that appear to be conserved in the fly. Certain categories such as cancer genes (72%) or genes involved in neurological disorders (64%), seemed to be better represented.

The identification of genes involved in neurodegeneration is a crucial step in the development of efficient therapeutic and diagnostic strategies. Pioneer work carried out by Seymour Benzer and colleagues, who screened for mutants with reduced lifespan and then examined them for signs of degeneration, demonstrated the feasibility of the approach [Curr.Biol. 7 (11): 885. 1997; Kretzschmar D et.al., (1997) The swiss cheese mutant causes glial hyperwrapping and brain degeneration in Drosophila. J Neurosci 17: 7425-7432; Buchanan R L et.al., (1993) Defective glia in the Drosophila brain degeneration mutant dropdead. Neuron 10: 839-850; Trends Genet. 16 (4): 161. 2000]. With a similar intention, Kretzschmar et.al. screened mutants with morphological defects in the adult brain using head sections [Bettencourt da Cruz et.al. (2005) Disruption of the MAP1B-related protein FUTSCH leads to changes in the neuronal cytoskeleton, axonal transport defects, and progressive neurodegeneration in Drosophila. Mol Biol Cell 16: 2433-2442; Tschape J A et.al. (2002) The neurodegeneration mutant lochrig interferes with cholesterol homeostasis and Appl processing. EMBO J 21: 6367-6376]. This type of approaches is clearly time-consuming and limited to the identification of genes causing severe defects in the anatomy of the adult brain. Ganetzky et.al., on the other hand, performed a more “physiological” screening in the search for histological signs of degeneration in mutants originally isolated for presenting paralytic phenotypes. This work is based on the notion that neuronal dysfunction, which causes quantifiable behavioral phenotypes, is often associated with neurodegeneration [Palladino M J et.al., (2002) Temperature-sensitive paralytic mutants are enriched for those causing neurodegeneration in Drosophila. Genetics 161: 1197-1208; Palladino M J et.al., (2003) Neural dysfunction and neurodegeneration in Drosophila Na+/K+ ATPase alpha subunit mutants. J Neurosci 23: 1276-1286].

In the last few years, several mutants have been isolated which cause a variable degree of neurodegenerative phenotype. These can be artificially classified as those involved in the maintenance of the structure and function of the nervous system like drop dead [J.Neurosci. 17 (19): 7425. 1997), swiss cheese (Proc.Natl.Acad.Sci.U.S.A 101 (14): 5075. 2004; 8. Neuron 10 (5): 839. 1993), and futsch (Mol.Biol.Cell 16 (5): 2433. 2005] or, alternatively, those which play a role in crucial metabolic functions, such as the response to oxidative stress such as for example, sniffer [Curr.Biol. 14 (9): 782. 2004]. In this sense, benchwarmer has been identified [J.Cell Biol. 170 (1): 127. 2005] as involved in storage in lisosomes and lochrig [EMBO J. 21 (23): 6367. 2002] in the metabolism of lipids. Min and Benzer (1997) performed a screening with alkylating agents (of the ethylmethanesulfonate type, or EMS) for tracing those relevant mutants in the shortage of life expectation in the fly, and reported the identification of spongecake and eggroll, which contain inheritable mutations causing a specific pattern of neuronal degeneration [Min K T et.al., (1997) Spongecake and eggroll: two hereditary diseases in Drosophila resemble patterns of human brain degeneration. Curr Biol 7: 885-888]. The brain of spongecake aged mutants shows vacuolization at specific sites, having a similar appearance to the ones observed in spongiform degenerations of the axonal terminals which are typical of the Creutzfeld-Jakob's disease. On the other hand, eggroll generates opaque, multi-lamellar structures, which look like those characteristic of lipid storage diseases such as Tay-Sachs's disease.

Patent documents U.S. Pat. No. 6,943,278, U.S. Pat. No. 6,489,535, U.S. Pat. No. 7,060,249 and WO 03/065795 disclose several transgenic Drosophila models for the study of neurodegenerative phenotypes.

As a consequence, although there have been identified diverse neurodegenerative mutants along time, given that the Drosophila genome contains more than 15000 genes, there is still a need for having a method that allows for a systematic genetic screen for the identification of novel genes potentially relevant in neurodegenerative processes which are characterized by a late onset and progressive degeneration, such Alzheimer's disease, Parkinson's disease and Huntington's disease.

### SUMMARY OF THE INVENTION

It is therefore an object of the present invention to provide a method for the identification of genes involved in neurodegeneration by means of a systematic genetic screen based on the assessment of a progressive behavioral phenotype as a function of time in young and aged transgenic animals carrying the same mutation.

According to preferred embodiments, the transgenic animals are invertebrate transgenic animals, particularly members of the phylum arthropods, and more particularly members of the class insecta. In a preferred embodiment the insects are flies, preferably transgenic flies that are members of the Drosophilidae family, for example Drosophila melanogaster.

According to an aspect of the invention, the inventors show that, abnormalities in the natural ageing pattern of the and rest/activity cycle, or, in other words, the loss of rhythmicity of the circadian cycle, will lead to the identification of genes involved in neurodegenerative processes.

Accordingly, the present invention provides a method for the identification of genes involved in neurodegenerative processes, detectable by the late onset of a phenotype associated with neurodegeneration, by means of a genetic screen of miss-expressed genes, which comprises the measurement of sleep-wake cycle activity schemes in different stages of life, young and adult, of individuals of an animal model, such as Drosophila, said method comprising the steps of:
i) assessing the standard rhythmicity of locomotor activity in alternate cycles of light and darkness conditions followed by a period of continuous darkness, of wild type non-mutant flies, at an early moment in life and at an intermediate stage in adult life;
ii) generating a collection of mutant flies by random insertional mutagenesis with a specific transposon, followed by crossing to a transgenic line comprising a tissue-specific neuronal expression promoter, which regulates the transcription factor with recognition sites in the transposon;
iii) assessing the rhythmicity of locomotor activity in alternate cycles of light and darkness conditions, followed by a period of constant darkness, in mutant flies generated in step (ii) at an early moment in life and at an intermediate stage in adult life;
iv) detecting and selecting the mutant individuals showing deviations with respect to the standard rhythmicity in said intermediate stage in adult life;
v) identifying the transposon insertion site; and
vi) identifying the gene trapped by said insertion.

According to an embodiment of the present invention, said early moment in life is a period comprised between 0 and 3 days of life, and said intermediate stage in adult life is a period comprised between 20 and 30 days of life.

According to an embodiment of the present invention, the genetic screen is based on the deregulation of genes restricted to a relevant circuit for the control of the rhythmic behavior that is not essential for life itself, and which is contrasted at two stages of life. More particularly, the insertional mutagenesis is directed to the deregulation of endogenous genes which are expressed within a restricted neuronal circuit controlling locomotor activity, underlying the circadian behavior, that is, after entrainment in alternate cycles of light and darkness. Yet more particularly, the step of generating a collection of mutant individuals comprises crossing a line resulting from the transposition of a MASI element with a transgenic line expressing the GAL4 transcription factor, under the control of a promoter of the gene encoding the pdf neuropeptide.

The neurodegeneration mutants identified in the method of the invention are valuable tools for the identification of proteins and key biochemical pathways required for the maintenance of neuronal viability. Therefore, according to another additional embodiment, the method according to the present invention further comprises identifying, based on publicly available data in the Internet, the human homologous genes identified in step (vi) of the method of the invention, described above.

As a consequence, the mutants identified in the method of the invention may be advantageously used for developing new therapies for treating and preventing neurodegenerative disorders in human and non-human animals.

Additionally, the mutants identified by the method of the invention constitute a valuable tool for its use in the in vivo screening of therapeutic agents potentially useful in the treatment of neurodegenerative disorders, particularly those related with human neurodegenerative diseases that are characterized by a late onset and progressive degeneration, such as Alzheimer's disease, Parkinson's disease and Huntington's disease. Said assessment may be performed by means of standard methodology known in the art [Dokucu et.al., Lithium- and valproate-induced alterations in circadian locomotor behavior in Drosophila, Neuropsychopharmacology (2005) 30, 2216-2224; Desai et.al., (2006), Biologically active molecules that reduce polyglutamine aggregation and toxicity, Hum. Mol. Genet. 15, 2114-2124.]. Specifically, the therapeutic agents are administered with the food to adult flies, thus avoiding potential teratogenic effects.

It is therefore an additional embodiment of the present invention a method for assessing a candidate compound for the treatment, prevention or therapeutic enhancement of neurodegenerative processes with late onset, characterized by comprising:
administering by the oral route said candidate compound to a mutant fly identified according to step (iv) of the method of the invention, and
comparing the changes in the phenotype of said mutant fly of the step above with the phenotype of a fly carrying the same mutation, to which no candidate compound has been administered,
   wherein the phenotype to be assessed is the rhythmicity of locomotor activity in alternate cycles of light and darkness conditions, followed by a period of constant darkness, at an intermediate stage of the adult life comprised between the 20 and 30 days of life.

According to an aspect of the present invention, a candidate mutant fly has been identified which shows progressive arrhythmicity with reduced expression levels of the enabled gene, a gene involved in active remodeling of actin cytoskeleton. The present inventors have demonstrated that reduced ena levels cause neuronal dysfunction, leading to progressive behavior abnormalities and neuronal death.

It is therefore an object of the present invention a fly whose genome comprises a disruption in its enabled gene, wherein said disruption strongly reduces the expression of the enabled gene, and said fly exhibits a late onset neurodegenerative phenotype in adulthood. Particularly, said late onset neurodegenerative phenotype in adult stage of life consists in the loss of rhythmicity of locomotor activity under free running conditions in the period of life comprised between 20 and 30 days of life.

Yet more particularly, it is an object of the present invention a mutant fly, the genome of which comprises a P[UAS] transposomal insertion which is located interrupting the first exon of the enabled gene, upstream of the ATG codon, which exhibits a late onset neurodegenerative phenotype in adulthood, which consists in the loss of rhythmicity of locomotor activity after synchronization in alternate cycles of light and darkness, in the life period comprised between 20 and 30 days of life.

According to another aspect of the present invention, a mutant fly has been identified which shows progressive arrhythmicity, and which genome comprises a P[UAS] transposomal insertion within the intergenic region between genes CG 15133 (recently renamed CG42555) and CG 6115, (CG: Celera Genome), said mutant fly exhibiting a late onset neurodegenerative phenotype in adult stage of life, wherein the late onset neurodegenerative phenotype in the adult stage of life consists in the loss of rhythmicity in locomotor activity in constant darkness, within the period of life comprised between 20 and 30 days of life. The present inventors have demonstrated that progressive arrhythmicity is accompanied by neurodegeneration in the adult brain.

### BRIEF DESCRIPTION OF THE DRAWINGS

FIG. 1A shows representative actograms from pdf-gal4/+ flies of increasing age showing two consecutive days (x axis) along time (y axis), wherein each panel depicts the activity of a single fly throughout the experiment. Age at the onset of the experiment is indicated at the bottom of each panel. White, grey and black boxes indicate day, subjective day and night, respectively; arrows represent the transfer to constant darkness; FIG. 1B shows the expression pattern of pdf-gal4 driving a UAS-CD8-GFP reporter gene in the adult brain; FIG. 1C shows a graph depicting the percentage of rhythmic flies for each genotype (CS and pdf-gal4+) as a function of age expressed in days.

FIG. 2A shows representative double plotted actograms of progressively older pdf>APP and control (pdf-gal4/+) flies; FIG. 2B shows a bar graph depicting the percentage of rhythmic flies for each strain (mutants pdf>APP and controls pdf-gal4/+); FIG. 2C shows a schematic diagram of the misexpression screen by means of the crossing between the pdf- gal4 line and a number of independent target P[UAS] lines; FIG. 2D shows a direct comparison of rhythmicity as the flies age, wherein those flies considered as potential neurodegenerative mutants (highly rhythmic when young but whose rhythmicity decreased severely as they aged) are indicated by .

FIG. 3A shows representative double plotted actograms for young (3 day-old) and aged (21 day-old) flies; FIG. 3B shows a bar graph depicting the percentage of rhythmic flies for each strain (controls pdf-gal4/+ and mutants pdf-gal4/P[UAS]¹¹⁷).

FIG. 4A shows a schematic diagram depicting the position of the P[UAS] transposon within the DNA region trapped by the insertion, for the genes ena, CG15111 and CG15118, wherein arrows indicate the direction of transcription for each gene; FIG. 4B shows images of the bands obtained by agarose gel electrophoresis stained with ethidium bromide, after performing 30 RT-PCR cycles using total RNA from hs>P[UAS]¹¹⁷ larvae after a heat-shock stimulus (+hs) and a non-pulsed control (−hs) as templates; FIG. 4C shows the quantification by RT-PCR of mRNA levels from different genes (ena, CG15111 and CG15118) in the hs>P[UAS]¹¹⁷ line (−hs and +hs).

FIG. 5A shows representative double plotted actograms for aged flies (24-28 day-old) from different genotypes (control UAS-ena/+; recombinant pdf-gal4,ena^{rev} carrying one copy of UAS-ena; and the pdf-gal4,ena^{rev}/++ strain); FIG. 5B shows a bar graph depicting the percentage of rhythmicity for aged flies of each strain of FIG. 5A; FIG. 5C shows representative actograms for young (3 day-old) and aged (21 day-old) flies of each genotype (control ena^{rev}/+, homozygous ena^{rev} and transheterozygotes ena^{rev}/ena^{GC5} flies); FIG. 5D shows a bar graph summarizing the behavioral data (rhythmicity) for flies of the genotypes indicated in FIG. 5C.

FIG. 6A shows single confocal planes (2 μm thick) at two depths (8 and 22 μm) of whole mount brain preparations of adult 10 day-old y w flies, studied by immunofluorescense analysis, stained with a specific antibody against ENA; FIG. 6B shows images taken with the same confocal settings as in FIG. 6A, for direct comparison of 2 to 3 μm depth projections; FIG. 6C shows the ratio between ena and actin expression levels for each genotype in adult flies (homozygous ena^{rev}, heterozygous ena^{rev}/+ and control y w) by RT-PCR quantification of RNA levels.

FIG. 7 shows frontal adult head semi-thin sections (1 μm thick) from flies of different genotypes (control elav-gal4/+, mutants elav>ena^{rev} containing the panneural promoter elav, and mutants th>ena^{rev} containing the th promoter which specifically drives GAL4 expression in dopaminergic neurons), stained with methylene blue and examined by light microscopy.

FIG. 8 shows frontal semi-thin head sections (1 μm thick) from flies of four different genotypes (ena^{rev}/+, ena^{rev}, c309>ena^{rev} and elav>P[UAS]²¹⁸), stained with methylene blue and examined by light microscopy.

FIGS. 9A1-9A4 show microscopy images of third-instar larval segmental nerves stained against CSP, a synaptic vesicle protein; FIG. 9B shows a bar graph of a quantitative analysis which measures clog density by cargo accumulation on segmental nerves from y w, elav>APP and elav>ena^{rev} larvae; FIG. 9C shows representative images of TUNEL staining from the y w, elav>APP and elav>ena^{rev} genotypes; FIG. 9D shows a quantitative analysis of TUNEL staining showing the extent of neuronal death in elav>ena^{rev}, positive controls elav>APP, and control line y w.

FIG. 10A shows a bar diagram of a quantitative analysis of apoptotic cell death in adult brains of increasing age, together with a representative image of brain in 30 day-old flies, shown on the upper left corner; FIG. 10B shows frontal brain sections (at approximately the same depth) of control aged flies (y w) and mutants elav>ena^{rev} with p35 and elav>APP; FIG. 10C shows representative actograms of aged lines pdf>ena^{rev}, p35 and control (left).

FIG. 11A shows representative double plotted actograms for young and aged flies of a control strain (pdf-Gal4/+) and a mutant strain (pdf-Gal4/P[UAS]^{100B}) . FIG. 11B shows a bar graph summarizing the percentages of rhythmicity for flies of the genotypes indicated in 11A. FIG. 11C shows an schematic diagram depicting the position of the P[UAS]^{100B} transposon within the DNA region trapped by the insertion.

FIG. 12 shows frontal semi-thin head sections (1 μm thick) from flies of different genotypes (control elav-gal4/+ and mutants elav>gal4/UAS-100B), stained with methylene blue and examined by light microscopy.

### DETAILED DESCRIPTION OF THE INVENTION

Drosophila has provided a powerful genetic system in which to elucidate fundamental cellular pathways in the context of a developing and functioning nervous system. Given that behavior provides a reliable readout of the state of the underlying neuronal circuit, and that neurodegeneration leads to early dysfunction of the circuits, the present inventors show that it is possible to identify components of the neurodegenerative processes by means of a genetic screen based on the assessment of the daily activity pattern in young and aged flies carrying the same mutation. Given that certain aspects of locomotion in flies decrease with ageing [Exp.Gerontol. 36 (7): 1137. 2001], the present inventors show that abnormalities in the natural ageing pattern of the activity and rest cycles will lead to identifying genes involved in neurodegenerative processes.

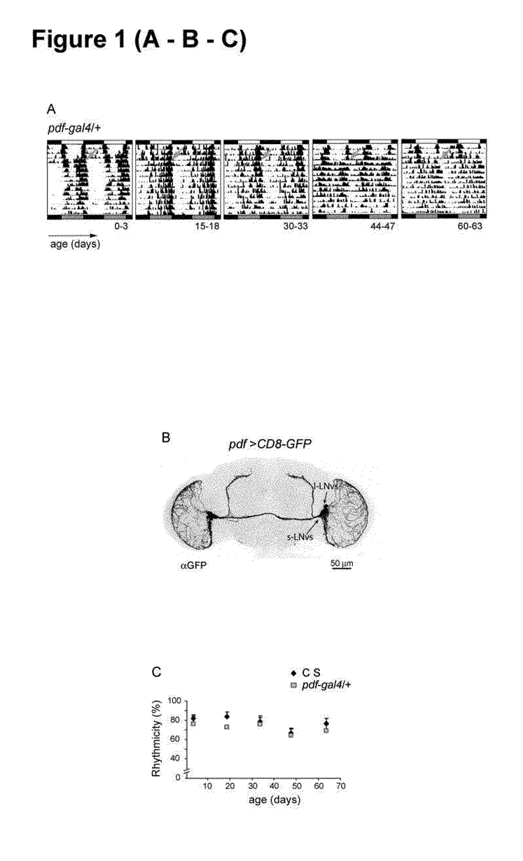

The extensive characterization of the neuronal circuit underlying circadian behavior makes it an ideal venue to search for mutations triggering neuronal dysfunction. This circuit includes eight neurons per brain hemisphere, four small and four large ventral Lateral Neurons (LNvs), which specifically express a neuropeptide called pigment dispersing factor (PDF, FIG. 1B) [Helfrich-Forster C (2003) The neuroarchitecture of the circadian clock in the brain of Drosophila melanogaster. Microsc Res Tech 62: 94-102]. It has been shown that this circuit is central to the control of rhythmic activity [Renn S C, et.al. (1999) A pdf neuropeptide gene mutation and ablation of PDF neurons each cause severe abnormalities of behavioral circadian rhythms in Drosophila. Cell 99: 791-802].

The history of circadian rhythms research shows the extraordinary advantage that phenotype-based screens may have in dissecting complex pathways such as those controlling rhythmic behavior [Proc.Natl.Acad.Sci.U.S.A 68 (9): 2112. 1971; Science 270 (5237): 805. 1995; Cell 93 (5): 791. 1998, among others]. Young flies are generally active around dawn and dusk. The present inventors apply this methodology for the comprehensive understanding of neurodegenerative processes, considering that progressive decline of the nervous system structures results in observable behavioral changes that directly or indirectly modify locomotor activity.

The identification of genes involved in neurodegeneration according to the present invention comprises, in the first place, the characterization of locomotor activity in wild type individuals, in order to be able to contrast with the emerging phenotypes of the mutant lines. Taking into account that observed neurodegeneration in patients suffering from neuropathologies is progressive in time, several control lines

(CS, y w and pdf-gal4;+) having increasing ages were analyzed. Lines y w, Canton-S, and pdf-gal4 were provided by the Bloomington Stock Center: y w (1495), C S (1), (6900) The recombinant line pdf-gal4+,ena^{rev} was generated in the lab by the present inventors. Drosophila cultures were maintained on a 12 hr light/dark cycle on standard corn meal yeast agar medium at 25° C. in an environmental chamber. Ageing flies were transferred into fresh vials every three days throughout the experiment.

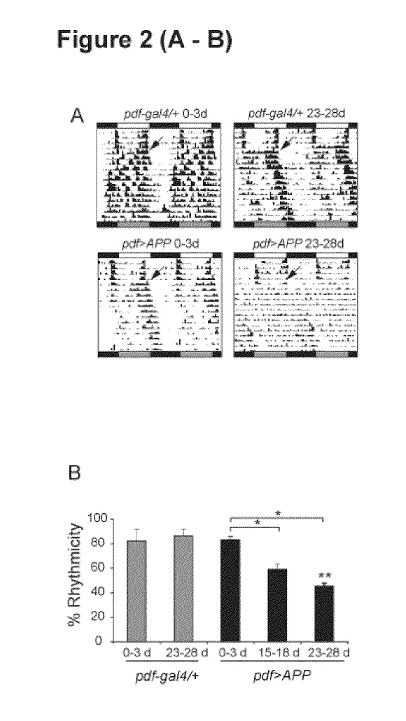

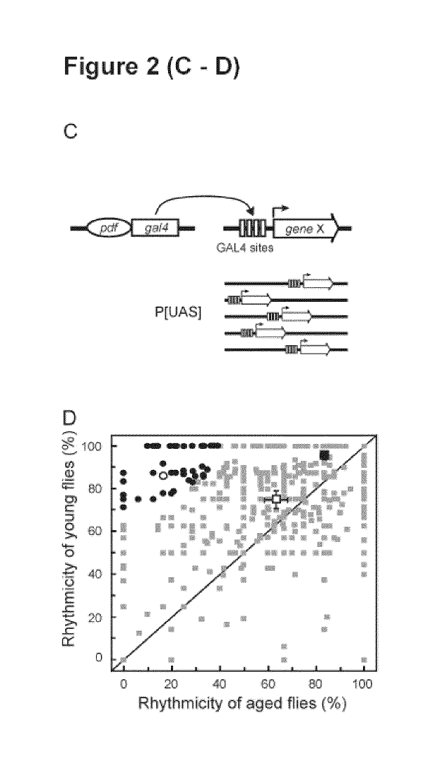

Mutants were generated by transposition of a P-element [Rorth P (1996) A modular misexpression screen in Drosophila detecting tissue-specific phenotypes. Proc Natl Acad Sci U S A 93: 12418-12422]. This mutant collection is characterized by containing the same P-element in different positions within the genome, and given that the insertion occurs at random (although there is a preference for inserting at 5′ non-codifying sequences (Proc. Natl. Acad. Sci. U.S.A 92 (24): 10824. 1995)), insertions could potentially be obtained in every gene. The P-element used is called UAS-hs and contains several binding sites for the GAL4 transcription factor in tandem (UAS), flanking the minimum promoter (i.e., incapable of driving transcription per se) of the gene codifying for a heat shock protein. The mutant collection is then crossed to a transgenic line expressing the GAL4 yeast transcription factor, which serves as a specific activator of the UAS sequence in Drosophila [Brand A H et.al., (1993) Targeted gene expression as a means of altering cell fates and generating dominant phenotypes. Development 118: 401-415], under the control of a desired promoter so as to force -in a controlled fashion- the expression of the gene adjacent to the P-element insertion site (FIG. 2C). In particular, the promoter of a gene encoding the pdf neuropeptide is used, which is constitutively expressed within a discrete group of neurons (the Lateral Neurons, NLs) which control the rhythmicity of locomotor activity [Biol.Rhythms 3 (3): 219. 1998], and are dispensable for life. This pdf-Gal4 line is used only in heterozygosis for avoiding problems associated with the excessive accumulation of GAL4, which may per se have a degenerative effect [Eur.J.Neurosci. 25 (3): 683. 2007].

The mutant flies resulting from each crossing were comparatively assayed, at the ages of 0-3 day-old (young) and of at least 21 day-old (aged). Activity of the flies was monitored under light/dark conditions for 4 days, after which they were left in the darkness for at least one week using commercially available activity monitors (Trikinetics, Walthman, Mass.). Activity of individual young (0-3 day-old) and aged (21 day-old) flies was examined. Period and rhythmicity were estimated using the Clocklab software (Actimetrics, Evanston, IL) from data collected in constant darkness. Flies with a single peak over the significance line in a Chi-Square analysis were scored as rhythmic, which was confirmed by visual inspection of the actograms. The FFT parameter represents the strength of rhythmicity. Flies classified as weakly rhythmic were not taken into account for average period calculations [Eur.J.Neurosci. 25 (3) : 683. 2007]. Total activity levels were determined as total counts per day displayed for each fly. Data shown in FIGS. 1, 3 and 5 were obtained from at least three independent experiments.

Once putative mutants were selected, the genes involved were identified. The transposon insertion site, and consequently the gene potentially responsible for the observed phenotype, is determined either by P-element rescue or by using the reverse PCR technique. Briefly, both techniques require the isolation of genomic DNA from the mutant of interest, which is digested with enzymes cutting towards an end of the P-element. This DNA is ligated so as to promote intracatenary reactions and is then used as a template for reverse PCR using specific primers, or for transforming E coli. Both strategies are complemented with sequencing of the flanking regions for determining the insertion site.

Knowing said sequence, identification of the genes in the region of influence is trivial, given that it only requires a simple comparison against the Drosophila genome (using databases and available software from the Internet). The complete sequence gene is obtained by RT-PCR from a total RNA adult head preparation, in the event that no EST (expressed sequence tags) is available at the public Stock Centers (Berkeley Drosophila Genome Project, for example).

In order to confirm whether the rescued gene is the one whose deregulation derives in the phenotype of interest, GAL4 is expressed in a generalized pattern to allow the detection over basal levels (using the heat shock promoter). Total RNA is extracted from mutants and controls, and a RT-PCR using specific oligonucleotides is performed for each one of the adjacent genes, for determining which of them is differentially expressed when compared to their respective controls. For completing this analysis, genetic interaction assays are performed, in which the effect of the genes flanking the insertion is examined, using mutants for each one of them available in the Stocks Centers (Bloomington, Szeged, Kyoto) in the behavioral paradigm. This strategy allows determining the effect of the partial loss-of-function for each gene (potentially affected by the insertion in the original mutant) in the context of the mutant under study. Comparison of the effect over behavioral rhythmicity in the transheterozygotes with respect to each insertion separately (i.e., in heterozygosis) allows determining whether other genes within the affected region contribute to the final phenotype. These experiments, not only will establish (or reject) the relevance of a particular gene in the deconsolidation of this behavior, but will also confirm that other mutations in the same gene (but in different genetic backgrounds, given that they originally derive from different collections) also lead to progressive dysfunction. This analysis controls from a potential genetic background effect, thus confirming that the phenotype observed may be unequivocally attributed to the specific deregulation of the gene of interest.

The neurodegeneration mutants identified in the method of the invention are valuable tools for the identification of proteins and key biochemical pathways required for the maintenance of neuronal viability. As a consequence, according to another additional embodiment, the method according to the present invention further comprises identifying, based on publicly available data in the Internet, the human homologous genes of the genes identified in the method of the invention, described above.

More particularly, the genes identified by the method of the present invention may be correlated to the human homolog genes, in order to elucidate the potential molecular function of the gene in question, as well as to identify the molecular pathways in which they are involved. Depending on the motifs identified in the Drosophila counterparts of the human genes (homologs), different molecular approaches could be deemed appropriate, such as: electrophoresis mobility shift assays or chromatin immunoprecipitations to test for ability to bind DNA, which when performed on genomic microarrays should help identify all potential targets in the genome; two hybrid assays in yeast or immunoprecipitations using tagged versions of the candidate proteins to inquire about potential interacting proteins, just to mention a couple of examples. In addition, fusion proteins with fluorescent tags (such as YFP or CFP) could be generated to address sub-cellular localization in transient or stable cell assays.

The following examples are provided in order to demonstrate and illustrate certain embodiments and preferred aspects of the present invention and should not be considered as limiting the scope thereof.

### EXAMPLES

### Example 1

### Identification of age-associated changes in circadian behavior

In order to identify progressive changes in circadian behavior, the pattern of rest/activity cycles at different times during adult life was examined in several Drosophila control lines, scoring a set of circadian parameters. FIG. 1A includes a representative actogram of progressively older heterozygous pdf-gal4 flies bearing a single copy of the driver employed in the genetic screen. The rest/activity cycles at different times during adult life examined for these control lines (pdf-gal4/+ flies of increasing age) may be observed in FIG. 1A. In the actograms, each panel depicts the activity of a single fly along the experiment. The age at the beginning of the experiment is indicated as a foot note below each panel. White, grey and black boxes indicate day, subjective day (i.e., day for those individuals kept at constant darkness conditions) and night, respectively; arrows represent the transfer to constant darkness.

Additionally, two commonly used wild type strains (Canton S and y w) were examined in parallel. Flies were synchronized in 12:12 h light/dark cycles for 4 days and then kept in constant darkness (DD). Free running behavior was monitored for 10 subsequent days. Period was calculated using the Clocklab package, by means of a Chi Square periodogram analysis, for which only rhythmic individuals were exclusively employed. Age and number (n) of analyzed individuals per genotype are indicated in Table I below. Percentage of rhythmic (R), weakly rhythmic (WR) and arrhythmic (AR) individuals are indicated. Also, average period, FFT average (FFT is a quantification which gives an idea of rhythm strength) and total activity of said individuals are indicated as well.

TABLE I

| | Age | | CD | | | Average | | Total |
|---|---|---|---|---|---|---|---|---|
| Genotype | (days) | n | % R | % WR | % AR | period (h) | FFT | activity |
| C S | 0-3 | 75 | 82.1 | 16.2 | 1.7 | 23.67 ± 0.05 | 0.15 ± 0.01 | 1566 ± 72.25 |
| | 15-18 | 80 | 83.6 | 12.6 | 3.8 | 23.77 ± 0.51 | 0.11 ± 0.01 | 1514 ± 74.89 |
| | 30-33 | 78 | 78.6 | 17.8 | 3.6 | 24.10 ± 0.06 | 0.12 ± 0.01 | 1543 ± 181.8 |
| | 44-47 | 66 | 66.8 | 24.0 | 9.2 | 24.02 ± 0.09 | 0.13 ± 0.02 | 1710 ± 99.73 |
| | 60-63 | 54 | 76.5 | 18.3 | 5.2 | 24.08 ± 0.08 | 0.10 ± 0.01 | 1098 ± 45.40 |
| pdf- | 0-3 | 66 | 76.0 | 17.5 | 6.5 | 23.89 ± 0.10 | 0.10 ± 0.01 | 1005 ± 60.89 |
| gal4/+ | 15-18 | 72 | 73.9 | 24.0 | 2.1 | 24.06 ± 0.10 | 0.10 ± 0.01 | 846.5 ± 46.58 |
| | 30-33 | 68 | 75.6 | 24.4 | 0.0 | 24.21 ± 0.10 | 0.10 ± 0.01 | 922.0 ± 52.95 |
| | 44-47 | 66 | 64.4 | 26.9 | 8.7 | 24.47 ± 0.10 | 0.08 ± 0.01 | 769.3 ± 70.72 |
| | 60-63 | 67 | 68.9 | 26.6 | 4.5 | 24.29 ± 0.09 | 0.08 ± 0.01 | 768.9 ± 50.17 |

Most parameters stayed relatively constant throughout flies' lifespan. Surprisingly, rhythmicity was only subtly affected as the flies aged (more than 30 days old); as can be seen in the actograms of FIG. 1A and the graph in FIG. 1C, exhibiting lack of consolidation of the bouts of activity during the next day (compare left and right actograms in FIG. 1A). However, this deconsolidation did not obscure the underlying rhythmicity assessed by periodogram analysis. Accordingly, the power of rhythmicity and the total locomotor activity tended to decrease in old files, while period length showed a tendency to increase reminiscent of what has been reported for other model systems [Joshi D et.al., (1999) Aging alters properties of the circadian pacemaker controlling the locomotor activity rhythm in males of Drosophila nasuta. Chronobiol Int 16: 751-758].

Hence, rhythmicity was selected as the readout (observable, measurable phenotype) for neurodegeneration-associated changes since although its age-related decrease is subtle, impairment of this neuronal circuit has a robust impact on this behavior [Fernandez M P et.al. (2007) Impaired clock output by altered connectivity in the circadian network. Proc Natl Acad Sci U S A 104:5650-5655]. Thus, three-week old flies were selected to search for progressive phenotypic alterations since wild-type flies display robust activity and rhythmicity at this stage (FIG. 1C).

### Example 2

### Selection of mutants showing a phenotype potentially involved in neurodegeneration by functional genetic screen (activity-rhythmicity patterns)

In order to identify genes involved in neurodegeneration through gene deregulation, without affecting the viability of the organism, the circadian system properties were altered by means of the transgenic line pdf-gal4 [Park J H et.al., (2000) Differential regulation of circadian pacemaker output by separate clock genes in Drosophila. Proc Natl Acad Sci U S A 97: 3608-3613] (FIG. 1B). To first test the notion that neurodegeneration could lead to progressive arrhythmicity, amyloid precursor protein (APP) expression was directed to the circadian circuit (pdf>APP). APP overexpression has been employed in fly models of Alzheimer's disease [Gunawardena S et.al., (2001) Disruption of axonal transport and neuronal viability by amyloid precursor protein mutations in Drosophila. Neuron 32: 389-401; Greeve I et.al., (2004) Age-dependent neurodegeneration and Alzheimer-amyloid plaque formation in transgenic Drosophila. J Neurosci 24: 3899-3906]; moreover, altered circadian patterns of activity have been reported in the APP23 mouse model, further strengthening this possibility [Vloeberghs E et.al., (2004) Altered circadian locomotor activity in APP23 mice: a model for BPSD disturbances. Eur J Neurosci 20: 2757-2766].

It is worth mentioning that when the rhythmicity of flies induced for APP overexpression (pdf>APP) was measured, a significant reduction was observed as the flies progressively aged, as may be seen in FIG. 2A, thus validating this behavioral readout. Three independent experiments were carried out, including forty to seventy flies. FIG. 2B shows the percentage of rhythmic flies for each strain. Aged pdf>APP flies showed reduced rhythmicity, which is significantly different from the respective controls.

Then, the pdf-gal4 line was employed to drive expression of independent transgenic insertions derived from a P[UAS] line carrying a transposable P-element [Rorth P, (1996)]. A simplified scheme of the misexpression construct is provided in FIG. 2C. The pdf-gal4 line was crossed to a number of independent target P[UAS] lines. In the progeny containing both elements, the GAL4 transcription factor binds to UAS within the P[UAS] transposon, inducing the misexpression of the gene immediately adjacent to it (gene X, in FIG. 2C).

Referring to FIG. 2D, a direct comparison of the degree of rhythmicity as flies age, i.e., newly eclosed and 3-week-old flies, was employed in order to identify genes potentially causing progressive neuronal dysfunction. The time frame was selected to ensure that most wild type flies would show no age-associated behavioral defects. Misexpression of most P[UAS] lines does not result in a progressive phenotype. Flies that were highly rhythmic when young but whose rhythmicity decreased severely as they aged were considered as potential neurodegenerative mutants and further retested (indicated by in FIG. 2D). Thus, it was observed that roughly ten percent of the misexpressed insertions displayed progressive defects in rhythmic behavior, whereby young flies were over seventy percent rhythmic and became arrhythmic by three weeks of age (highlighted in black in FIG. 2D).

The first stage in identification of mutations potentially related to neurodegeneration comprised the generation and screen of a collection of about 1000 insertional lines, generated by mutagenesis using a P-element as described above. Among the generated mutations, 30 preliminary targets were identified as causing a stronger behavioral defect in older ages, and the 8 mutants shown in Table II below were identified from them.

TABLE II

| Mutant | Trapped Gene | Known or predicted function |
|---|---|---|
| T117 | enabled | actin cytoskeleton remodeling |
| | CG15111 | ? |
| | CG15118 | ? |
| T100B | CG15133 | ? |
| | CG6115 | ? |
| T288 | CG3875 | binding to mRNA, transcription factor |
| | | associated to apoptosis |
| T303 | CG3919 | binding to DNA, transcription factor |
| | stonewall | binding to DNA, determination of oocyte |
| | | destiny |
| T11 | CG5050 | transcription factor? |
| T618 | rotated | protein glycosylation |
| | abdomen | |
| T338 | CG9171 | N-acetyl lactosaminide beta-1,6-N- |
| | | acetylglucosaminyltransferase |
| T821 | Btk29A | Tyrosine-protein kinase, determination of |
| | | life expectancy, sexual courtship, others. |

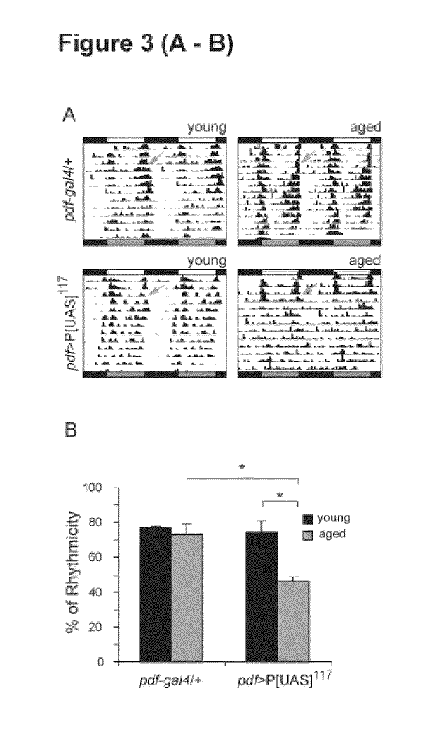

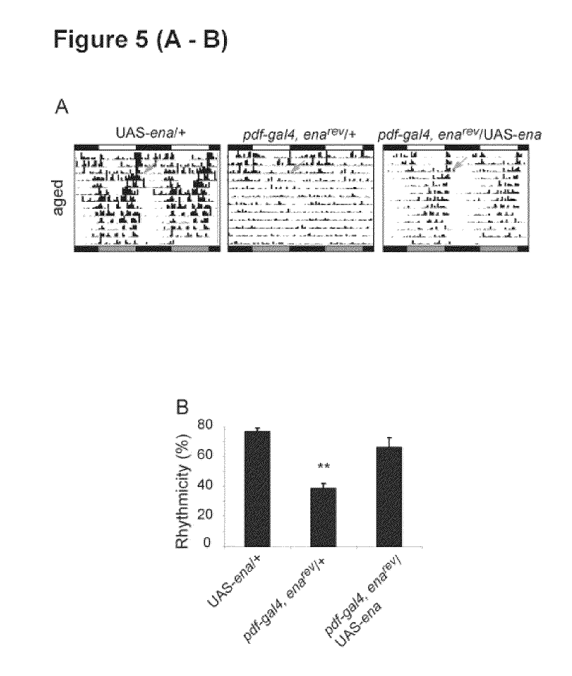

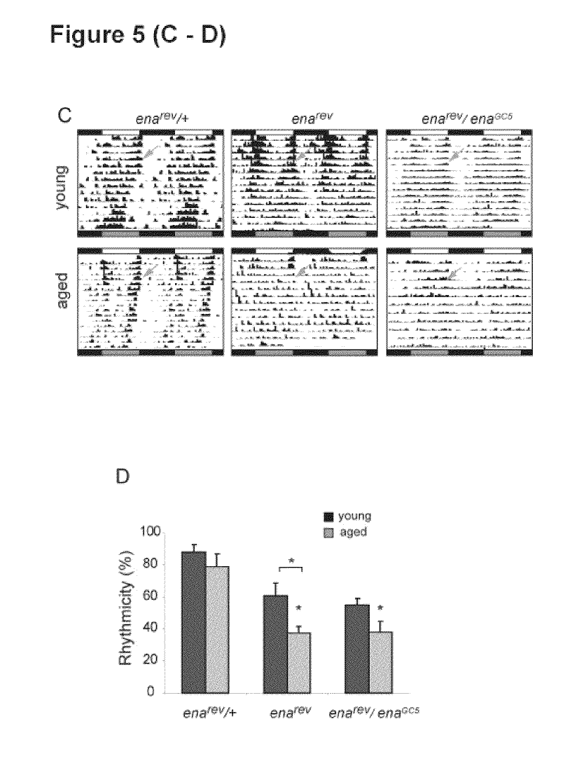

As can be seen from the actograms of FIG. 3A, obtained from representative young (3 day-old) and aged (21 day-old) flies, crossing P[UAS]¹¹⁷ to the pdf-gal4 driver resulted in a significant decrease in the rhythmicity of older flies. These insertions, which showed a robust age-dependent arrhythmicity, were selected for re-examination of the phenotype and further characterization. FIG. 3B shows the percentage of rhythmic flies for each strain. Older pdf-gal4/P[UAS]¹¹⁷ flies are significantly different than their younger counterparts and from the aged controls (*p<0.05). In particular, the pdf-gal4/P[UAS]¹¹⁷ line (from now on referred to as pdf>P[UAS]¹¹⁷) exhibited an age-dependent decrease in the percentage of rhythmicity, resulting from an abnormal deconsolidation of activity in subsequent days. This phenotype was not observed when analyzing in parallel a single copy of the pdf-gal4 driver (FIG. 3A-B) or the P[UAS]¹¹⁷ insertion in a heterozygous state (FIG. 5C-D).

These results suggest that GAL4 mediated alteration of the loci potentially affected by the insertion of the P[UAS]¹¹⁷ element progressively impaired neuronal function, giving rise to an age-dependent defective behavior.

### Example 3

### Determination of the P[UAS]¹¹⁷ insertion site and measurement of expression levels of the affected genes

The site of transposon insertion was identified by plasmid rescue. This procedure requires the preparation of genomic DNA from the P[UAS]¹¹⁷ line, which is subjected to digestion with a suitable restriction enzyme so that a single cut takes place within the transposon. Digested genomic DNA is ligated in such conditions so as to promote intracatenary reactions and then transformed into a competent Escherichia coli strain. Isolated colonies are selected and plasmidic DNA is prepared, which is then sequenced.

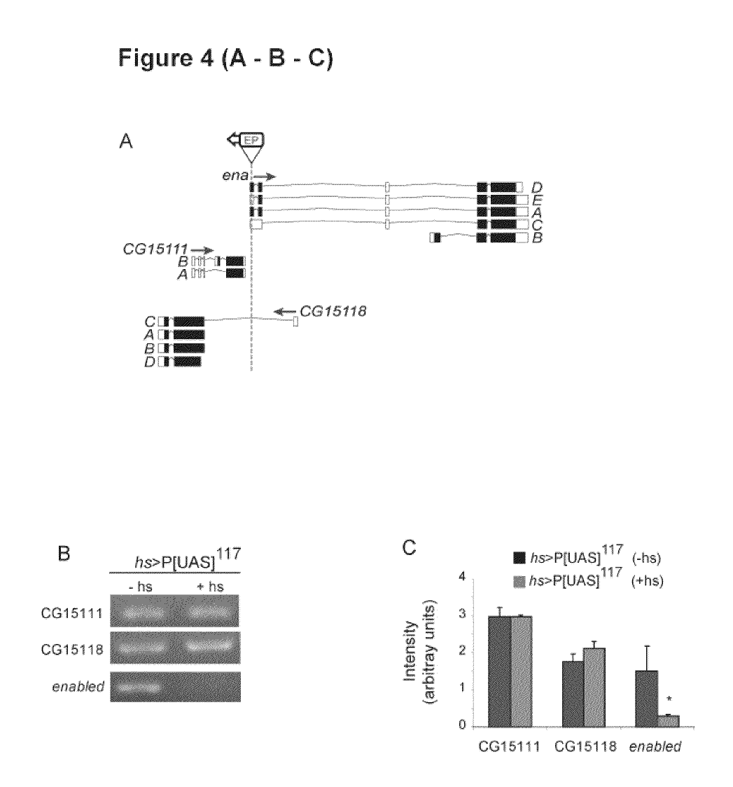

By means of said plasmid rescue analysis, it was revealed that P[UAS]¹¹⁷ element is inserted within the first exon of enabled (ena) upstream of the ATG, and thus it interrupts four out of the five splice variants predicted. FIG. 4A provides a schematic diagram depicting the position of the P[UAS] transposon within the DNA region interrupted by the insertion. The P[UAS]¹¹⁷ element also landed within the first intron of CG15118 and near CG15111. Arrows in FIG. 4A indicate the direction of transcription for each gene. The different splice variants in each loci are referred to as A-E.

The P element is observed to be located in reverse orientation with regard to transcription at the ena locus, potentially driving transcription of an antisense RNA in a GAL4-dependent manner. Such possibility is not unprecedented [Colombani J et.al., (2003) A nutrient sensor mechanism controls Drosophila growth. Cell 114: 739-749]. P[UAS]¹¹⁷ also interrupts the long splice variant of the gene CG15118; it is located within its first intron, upstream of the exon containing the ATG in the same orientation. The transcriptional start sites of the three remaining splice variants lie nearly 5 kb downstream, and therefore it is unlikely that they will be affected. Within this region there is a third predicted gene (CG15111) that runs in the opposite orientation to P[UAS]¹¹⁷ but it is not physically interrupted by it.

In order to identify the gene or genes potentially affected by GAL4 mediated expression the RT-PCR technique was employed. hs-gal4/ P[UAS]¹¹⁷ larvae of the strain selected in Example 2 were used, treated with a heat shock at 37° C. for 30 minutes (pulse) and then left at 25° C. for 2 hours for recovery, prior to their processing. This treatment (heat shock+recovery) was repeated twice. Non-pulsed controls were used for comparison.

Total RNA was isolated employing Trizol (Invitrogen). Reverse transcription was then performed using the SuperScript first-strand synthesis system (Invitrogen) according to the manufacturer's instructions. PCR analysis was carried out using the following primers: enaFw 5′-CCCTTGAAAAGCCCAAACAC-3′ (SEQ ID NO 1); enaRv 5′-CCGGGCCTGATTGTACTTC-3′ (SEQ ID NO 2); 15118Fw 5′-AGGAAGCTTCCAACGCTGGAGT-3′ (SEQ ID NO 3); 15118Rv 5′- CAAGAGGAATTTGCCGACGG-3′ (SEQ ID NO 4); 15111Fw 5′- TGTTCATCTCTGGCTGTCATCG-3′ (SEQ ID NO 5); 15111Rv 5′- CCTGACGTGATCCTTTACGGT-3′ (SEQ ID NO 6); actinFw 5′- GAGCGCGGTTACAGCTTCAC-3′ (SEQ ID NO 7); actinRv 5′- ACTCTTGCTTCGAGATCCACA-3′ (SEQ ID NO 8).

PCR products were analyzed on agarose gels stained with ethidium bromide. The RT-PCR analysis was performed on total RNA from adult hs-gal4/ P[UAS]¹¹⁷ specimens with or without heat pulse. The ratio between the expression levels for enabled, CG15111, 15118 and actin for each genotype was determined. The experiment was repeated three times employing independent RNA preparations.

RT-PCR analysis was carried out with primers directed to a region present in all splice variants for each gene. Results are shown in FIGS. 4B and 4C. RT-PCR products were analyzed on agarose gels stained with ethidium bromide (the image reflects ena levels on the 30^{th} cycle, see FIG. 4B). Actin levels were compared for quality control of the independent RNA preparations. Quantitation of these experiments is shown in FIG. 4C. P[UAS]117 appears to strongly and specifically affect ena levels, while little or no change was observed for CG15111 and CG15118 genes. Interestingly, heat-shocked flies (+hs) showed about one fifth of ena levels compared to non- pulsed controls thus confirming that GAL4-driven expression is triggering the decrease of endogenous ena levels. Therefore P[UAS]¹¹⁷ was renamed as ena^{reverse(rev)} to reflect that GAL4 mediated expression results in deregulation of the ena locus; when crossed to a GAL4 source such scenario gives rise to a tissue-specific hypomorphic mutation (partial loss of gene function).

### Example 4

### Study of the relationship between reduced ena levels and the progressive behavioral phenotype (arrhythmicity)

In order to determine whether ena downregulation by itself could be responsible for the progressive arrhythmicity, two complementary approaches were carried out.

Firstly, a copy of UAS-ena was introduced in pdf>ena^{rev} to assess whether increasing ena expression within the GAL4-mediated hypomorph is sufficient to rescue wild type behavior. Restoring ENA levels reduced the arrhythmicity of aged pdf>ena' which became undistinguishable from control flies. On the other hand, overexpression of ENA in young flies did not affect locomotor activity rhythms (data not shown). FIG. 5A shows actograms for aged (24-28 day-old) flies. As can be seen, recombinants pdf-gaL4, ena^{rev} carrying one copy of UAS-ena were undistinguishable from control UAS-ena. FIG. 5B shows the percentage of rhythmicity for aged flies for each strain. pdf-gal4, ena^{rev}/++ is significantly different from the control UAS-ena line (** p<0.001).

To test whether other strategies to decrease ena levels could also give rise to arrhythmic behavior, ena^{rev} effect on locomotor activity in the context of a well characterized null mutant (ena^{GC5}) was tested [Gertler F B et.al., (1995) enabled, a dosage-sensitive suppressor of mutations in the Drosophila Abl tyrosine kinase, encodes an Abl substrate with SH3 domain-binding properties. Genes Dev 9: 521-533]. If reduced ENA levels were the sole responsible for the phenotype, transheterozygotes ena^{rev}/ena^{GC5} should recreate the defects observed in homozygous ena^{rev} flies.

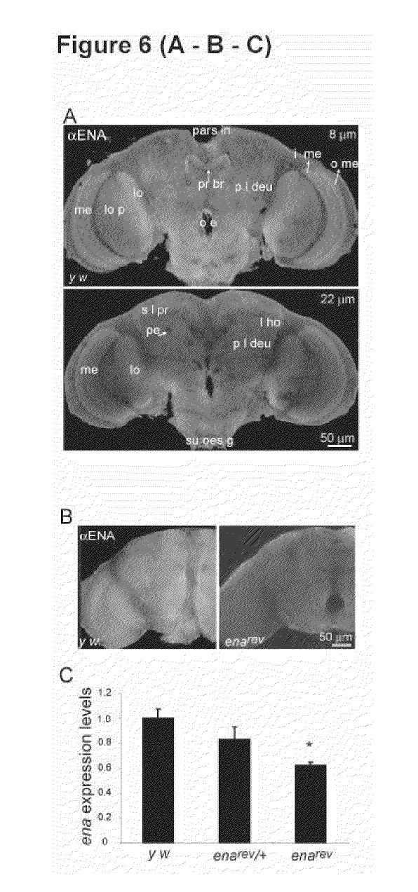

FIG. 5C shows representative actograms of young (3 day-old) and aged (21 day-old) flies carrying one or two copies of ena^{rev}, along with the transheterozygotes ena^{rev}/ena^{GC5}. Both ena^{rev} and ena^{rev}/ena^{GC5} exhibit a decline on rhythm strength. That is, ena^{rev} homozygote insertion per se showed a progressive decrease in the rhythmicity degree in older flies (FIG. 5C), probably due to a reduction in ena levels (FIG. 6C).

FIG. 5D summarizes the behavioral data (rhythmicity) for flies of the indicated genotypes. Control ena^{rev}/+ flies remained rhythmic throughout lifespan. Aged ena^{rev} (mutant) is significantly different from its younger counterpart (* represents p<0.05). Both aged ena^{rev} and ena^{rev}/ena^{GC5} are different from old ena^{rev}/+ (*p<0.05). Experiments summarized in B and D were repeated at least 3 times.

Referring again to FIG. 5C, progressive actograms are shown for ena^{rev}/ena^{GC5} transheterozygotes, phenocopying homozygous ena^{rev}, thus ruling out the contribution of unrelated loci potentially affected by the P-element insertion in ena^{rev}. Interestingly, both ena^{rev} and ena^{rev}/ena^{GC5} showed signs of deconsolidated activity as young adults. Neither ena^{GC5} nor ena^{rev} showed any defects when a single copy was examined (see FIG. 5C-D and Table III, below). Additionally, ena^{rev} was tested in the context of a P-element insertion that specifically affects CG15118 (stock 18105 from Bloomington Stock Center), to assess whether a higher impact on its levels could contribute to the observed phenotype: aged 18105/ena^{rev} individuals were highly rhythmic, as shown in the following Table III, thus ruling out a potential involvement of this locus in the behavioral phenotype.

TABLE III

| | Age | | |
|---|---|---|---|
| Genotype | (days) | n | % R (OO) |
| pdf-gal4/+ | 0-3 | 66 | 77.2 |
| pdf-gal4/+ | 21 | 54 | 73.2 |
| pdf > ena^{rev} | 0-3 | 55 | 74.4 |
| pdf > ena^{rev} | 21 | 89 | 46.2 |
| ena^{rev}/+ | 0-3 | 36 | 88.0 |
| ena^{rev}/+ | 21 | 55 | 78.9 |
| ena^{rev} | 0-3 | 40 | 60.4 |
| ena^{rev} | 21 | 86 | 37.4 |
| ena^{rev}/ena^{GC5} | 0-3 | 36 | 55.1 |
| ena^{rev}/ena^{GC5} | 21 | 51 | 38.1 |
| ena^{GC5}/+ | 21 | 18 | 89.6 |
| UAS-ena/+ | 24-28 | 30 | 76.4 |
| pdf-gal4, ena^{rev}/++ | 24-28 | 71 | 38.2 |
| pdf-gal4, ena^{rev}/UAS-ena | 24-28 | 60 | 66.1 |
| 18105/+ | 0-3 | 21 | 92.9 |
| 18105/+ | 21 | 41 | 90.9 |
| 18105/ena^{rev} | 0-3 | 46 | 100.0 |
| 18105/ena^{rev} | 21 | 37 | 84.8 |

Note:
Flies were synchronized and examined in the behavioral paradigm as indicated in Table I.

Summing up, this data supports the notion that progressive arrhythmicity derives from downregulated ena levels.

### Example 5

### Ena detection in the adult brain

As mentioned above in the present invention, enabled encodes a protein that links signaling pathways to the remodeling of actin cytoskeleton, and therefore is crucial for a variety of cellular process including morphogenesis, cell migration and adhesion [Krause M. et.al., (2003) Ena/VASP proteins: regulators of the actin cytoskeleton and cell migration. Annu Rev Cell Dev Biol 19: 541-564]. As such it has been implicated in axon pathfinding during nervous system development [Gertler F B et.al., (1995)]. However, a role for ENA in the adult brain has never been addressed.

In order to determine whether ena is expressed in the adult brain, an immunofluorescence analysis was carried out on whole mount brains employing an anti-ENA specific monoclonal antibody [Bashaw G J et.al., (2000) Repulsive axon guidance: Abelson and Enabled play opposing roles downstream of the roundabout receptor. Cell 101: 703-715].

To this end, the brains of ten day-old adult y w flies were dissected and then fixed in 4% paraformaldehyde in PB (100 mM KH₂PO_{4,}/Na₂HPO₄) between 30 minutes and 1 hour at room temperature. The excess fixative was removed by rinsing three times in PT (PBS plus 0.1% Triton X-100). Brains were then blocked in 7% goat serum in PT for 2 hr at room temperature. After the blocking step tissue was incubated with the primary antibody for 72 h at 4° C., and then washed for three times with PT for 20 minutes prior to the addition of the secondary antibody. After a 2 h incubation step, brains were washed for three times in PT and mounted in 80% glycerol (in PT).

The primary antibodies used were mouse anti-ENA (⅕, Developmental Studies Hybridoma Bank) or chicken anti-GFP ( 1/500, Upstate technologies). The secondary antibodies used were donkey Cy3-conjugated anti-mouse, Cy2-conjugated anti-chicken ( 1/250, Jackson ImmunoResearch) and Alexa 594 anti-mouse ( 1/250, Invitrogen). Detection of ENA in the adult brain was repeated at least three times examining 8-10 brains in each experiment. To compare ENA levels between wild type and mutant brains confocal fluorescence images were taken under the same conditions. A confocal Zeiss LSM510 microscope was used to image whole adult brains and larval preparations.

A homogenous ENA signal localized in several neuropils was observed, which resembles those expressing synaptotagmin [Littleton J T et.al., (1993) Expression of synaptotagmin in Drosophila reveals transport and localization of synaptic vesicles to the synapse. Development 118: 1077-1088]. FIG. 6A shows single confocal planes (2 μm thick) at two depths (8 and 22 μm) to highlight different brain areas. Some of the neuropils labeled with ENA are the outer (o me) and inner medulla (i me), lobula (lo) and lobula plate (lo p) within the optic lobe, the protocerebral bridge (pr br) in the central body complex as well as other regions in the protocerebrum such as the lateral horn (l ho). Other structures, as the protolateral deutocerebrum (p l deu), the peduncles (pe), pars intercerebralis (pars in), suboesophageal ganglion (su oes g) and oesophagus (oe) are also shown in the figure. As can be seen in FIG. 6A, primary sensitive centers such as the visual lamina (lamina, medulla, lobula and lobula plate in the optic lobe) were stained, as well as some central regions of the brain, including the central complex (such as, for example, the protocerebral bridge).

Immunohistochemistry analyses are shown in FIG. 6B (microscopy images). There, it can be seen that ena levels are reduced in ena^{rev} mutants compared to the control y w. Images were taken with the same confocal settings for direct comparison; projections of 2.3 μm depth are shown. ENA immunohistochemistry assays were repeated at least three times.

Referring to FIG. 6B, the immunohistochemistry analysis revealed that ENA expression was strongly reduced in homozygote ena^{rev} adults. In turn, a RT-PCR analysis on total RNA from ena^{rev}, ena^{rev}/+ adults and control (y w line), indicated that the ena^{rev} homozygous shows a significant reduction in ena expression while a single P[UAS]¹¹⁷ copy (such as in the ena^{rev}/+ mutant) resulted in a slight decrease in ena levels, which is consistent with its lack of effect over the behavioral paradigm (see FIG. 5C-D), which was confirmed by Western blot analysis (data not shown).

The ratio between ena and actin expression levels for each genotype is shown in FIG. 6C. As indicated above, quantification of RNA levels showed significant changes in ena^{rev} homozygous (*p<0.05) whereas a minor (non significant) decrease was seen in ena^{rev}/+ heterozygous when compared to the control line used. The experiment was repeated three times employing independent RNA preparations.

Detection of ENA in the adult brain indicates that this protein is present throughout the life of the organism, and thus its down-regulation could be triggering accumulative defects that in time result in behavioral impairment.

### Example 6

### Determination of the effect of ENA down-regulation in the adult brain and its relationship with progressive degeneration

In order to address whether down-regulated ENA function could lead to degeneration within the brain, two different drivers were employed: the panneuronal driver elav [Lin D M et.al., (1994) Ectopic and increased expression of Fasciclin II alters motoneuron growth cone guidance. Neuron 13: 507-523] and the th-gal4 promoter [Friggi-Grelin F et.al., (2003) Targeted gene expression in Drosophila dopaminergic cells using regulatory sequences from tyrosine hydroxylase. J Neurobiol 54: 618-627], which drives GAL4 expression specifically in the dopaminergic neurons.

The use of these promoters allows reducing ENA levels and thus permits to analyze its function in relation to neurodegeneration. In particular, to rule out potential artifacts due to region-specific expression levels associated to the elav-gal4, ENA misexpression was targeted to the dopaminergic neurons (employing th-gal4).

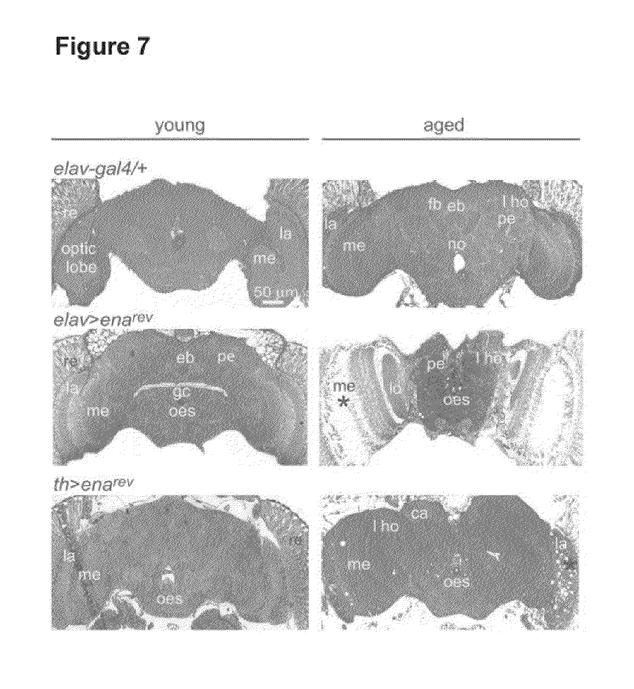

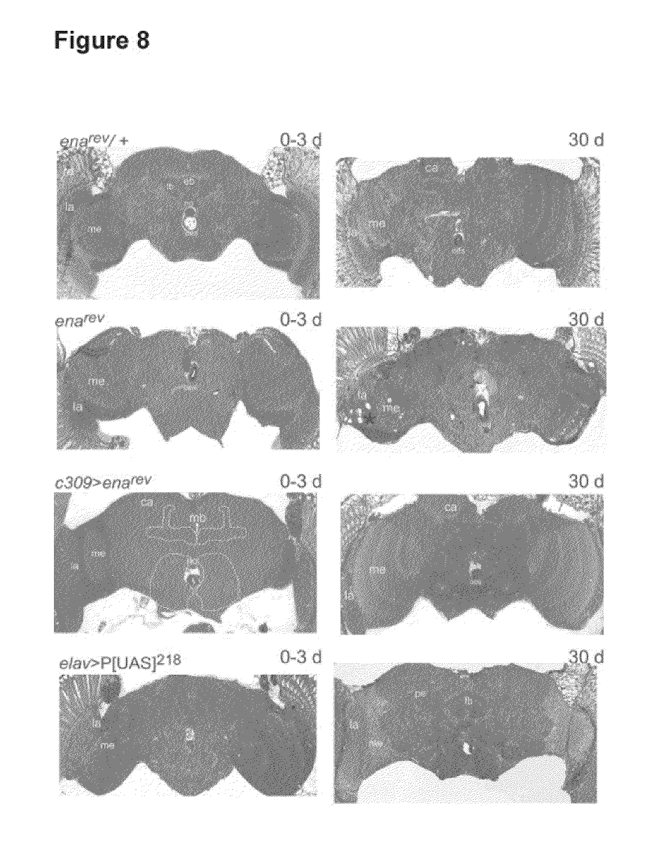

To perform this analysis, the procedure was as follows: frontal adult head semi-thin sections (1 μm thick) were stained with methylene blue and examined by light microscopy. Young (0-3 day-old) and old flies (30 day-old) were analyzed for each genotype. Heads were fixed with 3% glutaraldehyde in PBS for 2 h at room temperature, treated for 1-2 h in 1% osmium, dehydrated through several ethanol-steps and embedded in Spurr's epoxy resin. Four to ten heads from 0-3 or 30 day-old flies were analyzed per genotype in different trials occasions. Intermediate-age flies were examined for certain genotypes. Sections were visualized in a BX-60 Olympus microscope and photographed with a CoolSnap Pro digital camera. Images of the studied sections are shown in FIG. 7 and FIG. 8.

It was observed that reduction of ENA levels both panneurally and in the dopaminergic system caused degeneration in the same areas of the brain. As can be seen in FIG. 7, elav>ena^{rev} flies show age dependent vacuolization in the medulla and the lamina within the optic lobe while the nervous system of the control line (elav-gal4/+) is well preserved throughout the time evaluated. Control individuals, even aged, do not show signs of degeneration. Reduced ENA levels exclusively in dopaminergic neurons (th>ena^{rev}) also led to vacuolization in the optic lobe in aged flies, although to a lower extent.

Cortex and neuropil vacuolization verified in mutant brains (elav>ena^{rev}) was not evident in parental strains elav-gal4/30 and heterozygous ena^{rev} or in young elav>ena^{rev} flies, revealing an age-dependency of the neuropathological phenotype (see FIG. 7 and FIG. 8). On the other side, vacuolization in elav>ena^{rev} brains was not widespread. On the contrary, specific regions such as the medulla and the lamina in the optic lobe were particularly vulnerable to deregulated ENA, which is also supported by the observations made in ena^{rev} homozygous mutants (FIG. 8).

Interestingly, even though dopaminergic neurons are scattered throughout the adult brain, in th>ena^{rev} only the optic lobe showed clear vacuolization, although to a lower extent when compared to elav>ena^{rev}. Moreover, ena misexpression in regions other than the optic lobe did not trigger any sign of neuronal death (an example with the C309>ene^{rev}mutant [Kitamoto T (2002) Conditional disruption of synaptic transmission induces male-male courtship behavior in Drosophila. Proc Natl Acad Sci U S A 99: 13232-13237.] is shown in FIG. 8). The fact that the somatas of the small LNvs are located within a region highly vulnerable to ena misregulation likely accounts for the behavioral phenotype; in fact, the total number of PDF reactive neurons is reduced in 3 weeks old pdf>ena^{rev} flies (data not shown).

Taken together, these observations demonstrate that reduced ena levels cause neuronal dysfunction, leading to progressive behavioral abnormalities and neuronal death.

### Example 7

### Reduced ena levels trigger axonal transport defects

Fast-axonal transport cargoes, such as vesicle-associated synaptic terminal proteins and mitochondria, can accumulate in axonal swellings derived from mutation of kinesin 1 or dynein [Hurd D D et.al. (1996) Kinesin mutations cause motor neuron disease phenotypes by disrupting fast axonal transport in Drosophila. Genetics 144: 1075-1085; Gindhart J G, Jr. et.al. (1998) Kinesin light chains are essential for axonal transport in Drosophila. J Cell Biol 141: 443-454; Martin M y col (1999) Cytoplasmic dynein, the dynactin complex, and kinesin are interdependent and essential for fast axonal transport. Mol Biol Cell 10: 3717-3728; Bowman A B et.al. (1999) Drosophila roadblock and Chlamydomonas LC7: a conserved family of dynein-associated proteins involved in axonal transport, flagellar motility, and mitosis. J Cell Biol 146: 165-180]. ENA has been found to directly interact with kinesin heavy chain (Khc), a molecular motor involved in fast axonal transport [Martin M et.al. W M (2005) Abl tyrosine kinase and its substrate Ena/VASP have functional interactions with kinesin-1. Mol Biol Cell 16: 4225-4230.0]

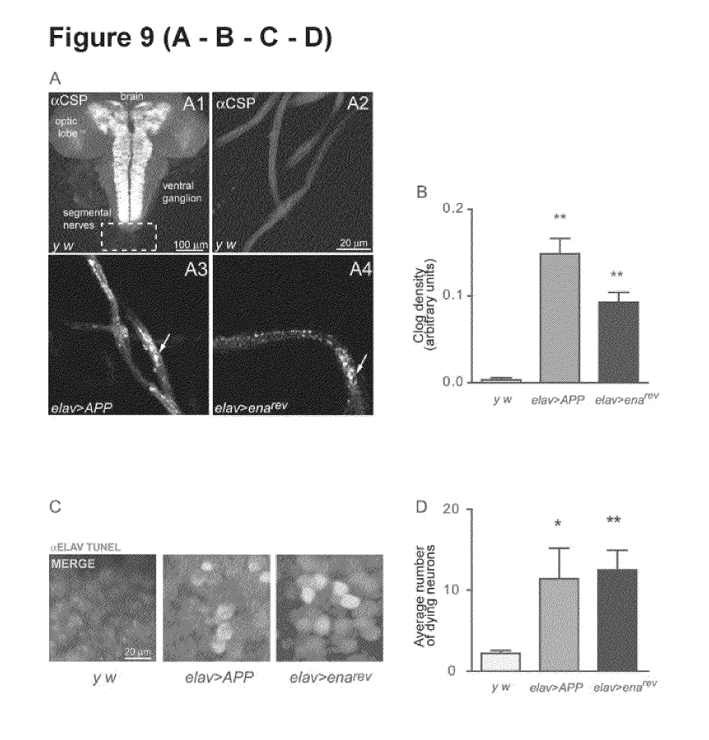

To examine whether ENA down-regulation could give rise to abnormal cargo accumulation, the localization of synaptic vesicle proteins CSP and SYT in the larval segmental nerves (see FIG. 9A1). To that end, larval brains from third-instar larvae were first removed preserving the segmental nerves in PBS, fixed in 4% formaldehyde PBS for one hour at 25° C. and then rinsed in PT. Samples were blocked in 7% goat serum in PT for 40 minutes at room temperature and then incubated with the primary antibody for 48 h at 4° C. Brains were then washed with PT for 40 minutes, followed by a 2 hour-incubation with the secondary antibody. After antibody staining, brains were washed three times with PT and mounted in 80% glycerol (in PT). Anti-REPO (glial marker) was used as neuronal specificity control. Primary antibodies used were anti-CSP, SYT and REPO at a final concentration of ⅕ (DSHB). Secondary antibodies were Cy2-conjugated goat anti-mouse IgG1 (1/250, Molecular Probes) and Cy5 conjugated goat anti-mouse IgG2b ( 1/250, Jackson ImmunoResearch).

FIG. 9 A1-A4 shows the immunohistochemistry of the preparations of intact brains from third-instar larvae, including larval segmental nerves (shown in the inset) corresponding to the genotypes indicated, which were stained against CSP, a synaptic vesicle protein. Axonal clogs are aggregates of membrane bound cargoes and can be a consequence of defective axonal transport [Hurd D D et.al. (1996)]. Segmental nerves from control larvae exhibit a relatively uniform CSP staining (FIG. 9A2).

Amyloid precursor protein (APP) overexpression (elav>APP) was included as a positive control, a manipulation that has already been demonstrated to induce axonal clogging [Gunawardena S et.al., (2001); Rusu P et.al. (2007) Axonal accumulation of synaptic markers in APP transgenic Drosophila depends on the NPTY motif and is paralleled by defects in synaptic plasticity. Eur J Neurosci 25: 1079-1086]. Consistent with this notion, the segmental nerves in elav>APP flies displayed conspicuous clusters of the presynaptic protein CSP (FIG. 9A3), which were absent in wild type controls (FIG. 9A2). Strikingly, reduced ENA levels in elav>ena^{rev} also resulted in the development of axonal clogs (FIG. 9A4), suggesting impairment at this level.

Quantitative analysis on larval segmental nerves was performed essentially as described in Gunawardena S et.al. (2001). Thus, clog density was measured. elav>ena^{rev} flies were significantly different from the wild type controls, similarly to what was seen for elav>APP (FIG. 9B), (**p<0.001).

Comparable results were obtained when the localization of SYT was analyzed (data not shown).

Earlier work has shown that APP misregulation leads to apoptosis [Gunawardena S, et.al. (2001)]. To investigate whether reduced ena levels could also trigger this mechanism, TUNEL staining (in situ staining of apoptotic nuclei) was performed on non-fixed larval brains according to the manufacturer's recommendations (Apoptag Plus Fluorescent Kit, Millipore). Colocalization with ELAV (a neuronal marker) was used as counterstain.

FIG. 9C shows representative images of TUNEL staining on the indicated genotypes. Quantitative analysis of TUNEL staining showing the extent of neuronal death in elav>ena^{rev} and positive controls are shown in FIG. 9D, both significantly different from a wild type control (*p<0.05, **p<0.001).

Strikingly, increased cell death correlated with continuous down-regulation of ena levels, suggesting that the abnormal organelle accumulations observed in the elav>ena^{rev} mutant results in apoptotic cell death.

Taken together these results are consistent with the notion that reduced ena levels cause transport dysfunction of certain specific cargoes, thus contributing to the degenerative phenotypes.

### Example 8

### Study of ena down-regulation associated with progressive apoptotic cell death

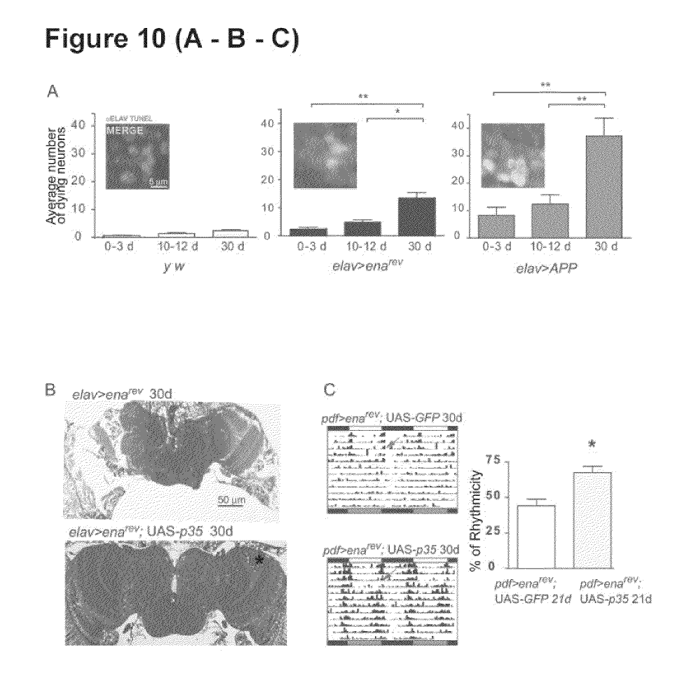

A quantitative analysis of apoptotic cell death was performed in adult brains of control flies (y w), mutants elav>ena^{rev} and elav>APP of increasing age. Results are shown in FIG. 10A. The extent of cell death in 30 day-old flies is shown in a representative image inserted in the upper left corner of each graph. The degree of apoptosis in the affected individuals is significantly different from a wild type control (*p<0.05, **p<0.001).

Reduced ena levels correlated with positive TUNEL staining in the larval brain; however young adult flies did not develop behavioral or anatomical defects. During metamorphosis the development of novel neuronal clusters and connections could generate a new architecture susceptible to ena down- regulation, which only in time would display such defects. In control brains a minimum level of TUNEL staining was observed, scattered throughout the brain, which did not significantly increase in older flies (FIG. 10A).

However, when mutant elav>ena^{rev} brains were stained, an increasing number of apoptotic neurons in the optic lobe was observed, albeit to a lower level than after APP overexpression. This data is consistent with a scenario in which reduced ena levels lead to neuronal dysfunction and eventually trigger apoptosis, in time affecting a larger and differentially susceptible neuronal population, thus accounting for the progressive behavioral and anatomical defects.

Also, in order to evaluate whether the extensive vacuolization observed in aged individuals derived solely from apoptotic cell death, an analysis of frontal head sections (at approximately the same depth) was carried out in the aged control and elav>ena^{rev}. To this end, a single copy of p35, a general caspase inhibitor [Hay B A et.al., (1994) Expression of baculovirus P35 prevents cell death in Drosophila. Development 120: 2121-2129], was introduced in elav>ena^{rev}.

Remarkably, most of the aged elav>ena^{rev}/p35 mutant brains displayed no vacuolization, while only a few showed vacuoles located in the most susceptible regions (FIG. 10B). The sections in FIG. 10B highlight the extent of the morphological rescue. The asterisk in the upper right corner of the image corresponding to elav>ena^{rev};UAS-p35 denotes a region where small vacuoles can still be found in one of the few brains in which the rescue was not complete.

On the other side, FIG. 10C shows the functional rescue of ena-derived behavioral phenotypes. Representative actograms of old pdf>ena^{rev}/p35 and control lines are included (left). The percentage of rhythmic individuals is also shown (right, *p<0.05). The rescue of arrhythmicity observed in pdf>ena^{rev}/p35 flies highlights that, regardless of additional mechanisms underlying ENA-mediated neurodegeneration, programmed cell death is an important effector.

### Example 9

### Determination of P[UAS]^{100B} insertion site and measurement of expression levels of the affected genes

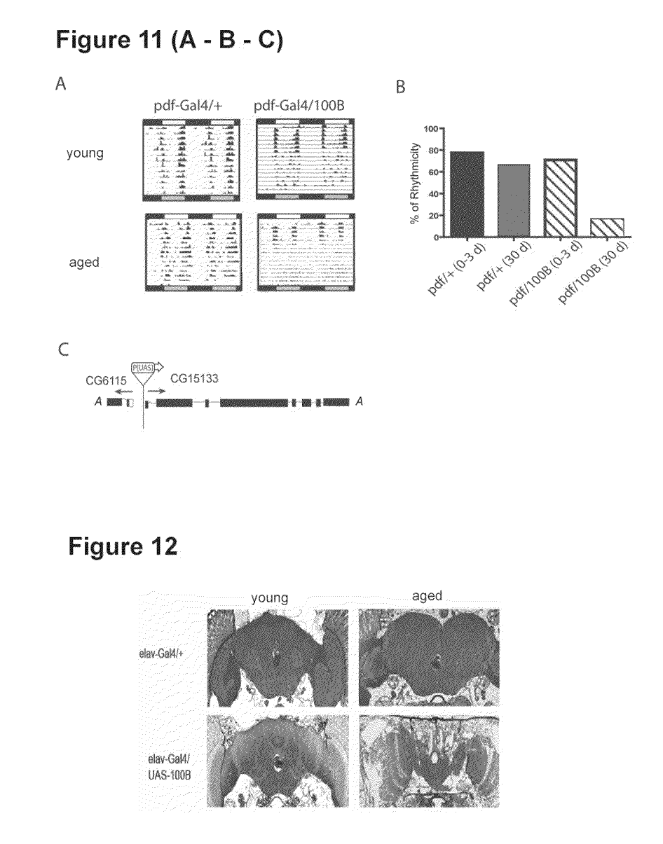

The site of transposon insertion was identified by plasmid rescue, as described in Example 3, from genomic DNA from 30 adult individuals of the P[UAS]^{100B} line. Even though this mutant does show a progressive arrhythmicity defect similar to P[UAS]¹¹⁷, the dysfunction caused results in a more severe effect over total locomotor activity (FIG. 11A). This mutant is lethal in homozygosis (manifested as lethality in larval instars L2 or L3, which suggests a central role at this developmental stage).

Plasmid rescue revealed that the P[UAS]^{100B} element is inserted in an intergenic region between the genes: CG 15133 (recently renamed CG42555) and CG 6115, both of unknown function.

The P element is located in the same orientation with regard to transcription in the CG15133 (CG42555) locus. P[UAS]^{100B} is located upstream to the transcription start site of the predicted gene for CG15133 (CG42555). Both transcript levels seem to be affected by the insertion, but only those from CG15133 (CG42555) are increased in the presence of GAL4 (data not shown). FIG. 11A shows a representative actogram of young and aged individuals of the genotypes pdf-Gal4/+ and pdf-Gal4/P[UAS]^{100B}. About 30 individuals per genotype were examined simultaneously in an average experiment. FIG. 11B shows the percentage of rhythmicity for the genotypes mentioned in a representative experiment. A clear decline is observed in the rhythmicity of aged pdf-Gal4/P[UAS]^{100B} individuals when compared to the controls. FIG. 11C shows a schematic diagram of locus organization indicating that the insertion is located between both genes. The Drosophila genome database only indicates one splice variant for each gene (“A”). Simple arrows indicate the direction of transcription for the corresponding loci, and the complex arrow indicates the transposon orientation, which would be mediating CG15133 overexpression through GAL4.

### Example 10

### P[UAS]^{100B} deregulation in the adult brain and its relationship with progressive degeneration

In order to elucidate whether the deregulation which leads to progressive behavioral arrhythmicity in P[UAS]^{100B} is also accompanied by degeneration in the adult brain, an analysis similar to that indicated in Example 6 was performed, employing the panneuronal driver elav [Lin D M et.al., (1994) Ectopic and increased expression of Fasciclin II alters motoneuron growth cone guidance. Neuron 13: 507-523].

As may be seen in FIG. 12, control individuals, even when aged, do not show signs of degeneration. On the contrary, those individuals in which the P[UAS]^{100B} levels are panneurally deregulated, show a remarkable vacuolization which mainly affects the neuropils involved in processing visual information, as well as more central areas of the brain (the central brain), which are responsible for the integration of information.

FIG. 12 shows representative images of head sections from young and adult flies. The images describe comparable regions of the brain from young and aged individuals for the genotypes indicated. P[UAS]^{100B} deregulation remarkably affects neuronal viability as derived from the extent of vacuolization typical of the mutants. It should be noted that young individuals of the same genotype do not show such signs.

1. A method for the identification of genes involved in late onset neurodegenerative processes by means of a genetic screen of deregulated genes in mutant flies, characterized by comprising:
i) assessing the standard rhythmicity of locomotor activity in alternate cycles of light and darkness conditions, followed by a period in constant dakrness, in wild type non-mutant flies, at an early moment in life and at an intermediate stage in adult life;
ii) generating a collection of mutant flies by random insertional mutagenesis with a transposon, followed by crossing to a transgenic line comprising a tissue-specific neuronal expression promoter, which regulates the transcription factor with recognition sites in the transposon;
iii) assessing the rhythmicity of locomotor activity in alternate cycles of light and darkness conditions, followed by a period in constant dakrness, in mutant flies generated in step (ii) at an early moment in life and at an intermediate stage in adult life;
iv) detecting and selecting the mutant individuals showing deviations with respect to the standard rhythmicity in said intermediate stage in adult life;
v) identifying the transposon site of insertion; and
vi) identifying the gene trapped by said insertion.

2. The method according to claim 1, characterized by the fact that the flies are Drosophila melanogaster flies.

3. The method according to claim 1, characterized by the fact that said early moment in life is a period comprised between 0 and 3 days of life, and wherein said intermediate stage of adult life is a period comprised between 20 and 30 days of life.

4. The method according to claim 1, characterized by the fact that the step of generating a mutant flies collection comprises crossing a line resulting from transposition of a P[UAS] element to a transgenic line expressing the GAL4 transcription factor, under the control of a promoter of the gene encoding the pdf neuropeptide.

5. The method according to claim 1, characterized by the fact that it further comprises identifying the human homologs of the genes identified in step (vi) of said method in databases publicly available in the Internet.

6. The method according to claim 1, characterized by the fact that the late onset neurodegenerative processes are processes which are manifested in the Alzheimer's, Parkinson's and Huntington's diseases.

7. Mutant fly characterized by the fact that its genome comprises a disruption in the enabled gene, wherein said disruption strongly reduced the expression of the enabled gene, and said fly exhibits a late onset neurodegenerative phenotype in the adult stage of life.

8. Mutant fly according to claim 7, characterized by the fact that the P[UAS] transposomal insert is located interrupting the first exon of the enabled gene, downstream to the ATG codon.

9. Mutant fly according to claim 7, characterized by the fact that the late onset neurodegenerative phenotype in the adult stage of life consists in the loss of rhythmicity of locomotor activity in alternate cycles of light and darkness conditions within the period of life comprised between 20 and 30 days of life.

10. Mutant fly characterized by the fact that its genome comprises a P[UAS] transposomal insert within an intergenic region between the genes CG 15133 and CG 6115, and said fly exhibits a late onset neurodegenerative phenotype in the adult stage of life.

11. Mutant fly according to claim 10, characterized by the fact that the late onset neurodegenerative phenotype at the adult stage of life consists in loss of rhythmicity of locomotor activity in alternate cycles of light and darkness conditions within the life period comprised between 20 and 30 days of life.

12. Mutant fly according to claim 7, characterized by the fact that it is a Drosophila melanogaster fly.

13. A method for assessing a candidate compound for the treatment, prevention or therapeutic enhancement of late onset neurodegenerative processes, characterized by the fact that it comprises:
administering by the oral route said candidate compound to a mutant fly identified according to step (iv) of the method of claim 1,
comparing the changes in phenotype of said mutant fly of the previous step with the phenotype of a fly carrying
the same mutation to which the candidate compound has not been administered,
wherein the phenotype to be examined is the rhythmicity of locomotor activity in alternate cycles of light and darkness conditions, at an intermediate stage of adult life comprised between 20 and 30 days of life.

14. The method of claim 13, characterized by the fact that it further comprises selecting the compound which restores the rhythmicity of locomotor activity in alternate cycles of light and darkness conditions, at an intermediate stage of adult life comprised between 20 and 30 days of life, of the fly to which the candidate compound was administered.

15. Mutant fly according to claim 10, characterized by the fact that it is a Drosophila melanogaster fly.
